(19) United States
(12) Plant Patent Application Publication
(10) Pub. No.: US 2003/0188365 P1
(43) Pub. Date: Oct. 2, 2003

(54) PETUNIA PLANT NAMED 'SUNBELBURA'

(76) Inventor: Yasuyuki Murakami, Gamo-gun (JP)

Correspondence Address: BURNS DOANE SWECKER & MATHIS L L P, POST OFFICE BOX 1404, ALEXANDRIA, VA, 22313-1404, US

(21) Appl. No.: 10/107,182

(22) Filed: Mar. 28, 2002

(51) Int. Cl. A01H 5/00

(52) U.S. Cl. PLT/356

(57) ABSTRACT

Disclosed herein is a new and distinct variety of Petunia plant having a decumbent habit. The Petunia plant has abundant branching particularly with respect to secondary branching, and great profusion of blooms, with the entire plant remaining in bloom for a considerable period of time. The flowers are very small with petals having a pale purplish pink color with deep reddish purple vein, and have a plentiful number of flower in a spike. The blooming period is from May to October. The plant exhibits high resistance to rain, cold, heat, pests and diseases.

### BACKGROUND OF THE VARIETY

The present invention relates to a new and distinct variety of petunia plant obtained as a mutation of petunia plant variety ‘SUNBELKUPI’ (registration No. 7230 under the Seeds and Seedlings Law of Japan).

The petunia is a very popular plant and is used for flower bedding and potting in the summer season. There are only a few varieties of the Petunia plants which do not have an upright growth habit and which have a high resistance to rain, heat, cold and diseases. Recently, we have developed series of small flower petunias, such as, ‘SUNTORY SP-R’ (U.S. Plant Pat. No. 9557), ‘SUNBERUBU’ (U.S. Plant Pat. No. 9754), ‘SUNBELOCHIPI’ (U.S. Plant Pat. No. 10,355) and ‘SUNBELKI’ (U.S. Plant Pat. No. 11,558) are of the semi-erect type, have short stems, a medium plant height, abundant branching, and a high resistance to heat, cold and rain. However, there are only a few varieties having a great profusion of flowers, a light yellowish pink with vivid reddish orange flower color, a very small flower size and a high resistance to rain, heat, cold and diseases. Accordingly, this invention was aimed at obtaining a new variety having a pale purplish pink with deep reddish purple flower color, and very small flowers combined with the above features.

The new variety of Petunia plant according to this invention originated from mutant occurring as a spontaneously generic variant of ‘SUNBELKUPI’ which we previously filed in Japan [‘SUNBELKUPI’ The Seeds and Seedlings Law Registration No. 7230] and the United States of America [PETUNIA PLANT NAMED ‘SUNBELKUPI’ (U.S. Plant Pat. No. 10,287)], and so on. The new variety of petunia plant was discovered in view of the flower color during propagation of ‘SUNBELKUPI’ in January of 2000 in the Omi Research Center of SUNTORY Ltd., at 863-1, Aza-Iketani, Omori-cho, Yokaichi-shi, Shiga-ken, Japan. The discovered petunia plant was propagated by cutting into 500 plants and then grown in pots on trial in the green house from January, 2001. The botanical characteristics of the plant were examined, using a similar variety, ‘Peal Sky Blue’ and the parent variety, ‘SUNBELKUPI’, for comparison. As a result, it was concluded that this petunia is distinguishable from any other variety whose existence is known to us, sufficiently uniform and stable in its characteristics, and this new variety of petunia plant was named ‘SUNBELBURA’.

In the following description, the color-coding is in accordance with the Horticultural Color Chart of The Royal Horticultural Society, London, England (R.H.S. Color Chart), Inter-Society Color Council—National Bureau of Standards (ISCC-NBS) and a color chart based on The Japan Color Standard for Horticultural Plant (J.H.S. Color Chart) is also added for reference.

‘SUNBELKUPI’, the mutant parent variety of ‘SUNBELBURA’, was obtained from crossing one petunia plant(♀), which was selected from crossing a wild type of petunia plant ‘A1’(♀) native to Brazil and another wild type of petunia plant called ‘B3’(♂), and a wild type of petunia plant called ‘B3’(♂) in January, 1993, in the Plant Biotechnology Laboratory, Institute for Fundamental Research of SUNTORY Ltd., at 1-1, Wakayamadai 1-chome, Shimamoto-cho, Mishima-gun, Osaka-fu, Japan.

A similar variety to the new variety ‘SUNBELBURA’ is ‘Pearl Sky Blue’, which was used for examination as a comparison. The main botanical characteristics of ‘Pearl Sky Blue’ are as follows.

Plant:

Growth habit.—Medium upright.

Plant height.—14 cm.

Spreading area of plant.—15-17 cm in diameter.

Blooming period.—April to September in the southern Kanto area, Japan.

Stem:

Thickness.—2.7 mm.

Pubescence.—Dense.

Branching.—Abundant.

Length of internode.—1.3 cm.

Leaf:

Shape.—Elliptic.

Length.(average).—5.5 cm.

Width.(average).—3.5 cm.

Color.—Strong yellow green (R.H.S. color chart No. 144A, JHS color chart No. 3507).

Thickness.—0.5-0.6 mm.

Pubescence.—Dense.

Leaf attachment angle to stem.—Horizontal to droopy.

Flower:

Facing direction.—Upward.

Type.—Single.

Shape.—Funnel-shape, with five-fissured limb.

Shape of petal tip.—Obverse.

Waving of petal.—Weak.

Lobation of petal.—Shallow.

Diameter.—5.5 cm.

Color.—Petal: Strong purple (R.H.S. color chart No. 83C, JHS color chart No. 8310).

Bottom color of the corolla throat and the outside color of corolla tube.—Light yellow green (R.H.S. color chart No. 3D, JHS color chart No. 3304).

Reproductive organs.—1 normal pistil and 5 stamens.

Peduncle.—0.7-0.9 mm in thickness, and 2.7 cm in length.

Physiological and ecological characteristics.—Moderate resistance to cold, heat, diseases and pests. Strong resistance to rain.

We previously filed ‘SUNBELKUPI’, which is the mutant parent of the variety ‘SUNBELBURA’, in Japan [‘SUNBELKUPI’ The Seeds and Seedlings law Registration No. 7230]] and the United States of America [PETUNIA PLANT NAMED ‘SUNBELKUPI’ (U.S. Plant Pat. No. 10,287)], and so on. The botanical characteristics of the petunia plant ‘SUNBELKUPI’ are as follows.

Plant:

Growth habit.—Decumbent. The stems hang down when potted in a hanging pot.

Plant height.—5.8-6.1 cm.

Spreading area of plant.—The stems extends to a length of 18-19 cm from the base, and thus the spreading area of the plant is 78-83 cm in diameter.

Growth.—Very vigorous with abundant branching, a great profusion of blooms; the whole bush remaining in bloom for a considerable period of time.

Blooming period.—April to late October in the southern Kanto area, Japan. The plant shape does not change throughout this period.

Stem:

Thickness.—1.2 mm.

Pubescence.—Normal to few.

Branching.—Very abundant. In particular, secondary branching is very strong.

Length of internode.—1.3 cm.

Leaf:

Shape.—Lanceolate.

Length.—3.5 cm.

Width.—1.0 cm.

Color.—Dark olive green. (R.H.S. color chart No. 137B, JHS color chart No. 3707).

Thickness.—0.2-0.3 mm.

Pubescence.—Few.

Leaf attachment angle to stem.—Slanted upward to horizontal.

Flower:

Facing direction.—Slanted upward to horizontal.

Type.—Single.

Shape.—Funnel-shape, with five-fissured limb.

Shape of petal tip.—Medium.

Waving of petal.—Weak.

Lobation of petal.—Very shallow.

Diameter.—2.7-2.9 cm.

Color.—Petal: Vivid reddish purple (R.H.S. color chart No. 74A, JHS color chart No. 9207).

Bottom color of the corolla throat.—Brilliant greenish yellow (R.H.S. color chart No. 3A, JHS color chart No. 2905).

Outside color of corolla tube.—Pale yellow green (R.H.S. color chart No. 1D, JHS color chart No. 3102) with moderate olive lines (R.H.S. color chart No. 152B, JHS color chart No. 2710).

Reproductive organs.—1 normal pistil and 5 normal stamens. Both normal.

Peduncle.—0.7-0.8 mm in thickness, and 1.8 cm in length.

Physiological and ecological characteristics.—High resistance to rain, heat, drought and pest, resistance to rain and heat is especially strong.

Fertility.—Fertile but self-incompatible.

This new variety of Petunia plant ‘SUNBELBURA’ was asexually reproduced by cutting at the Omi Research Center of SUNTORY Ltd., at 863-1, Aza-Iketani, Omori-cho, Yokaichi-shi, Shiga-ken, Japan, and the homogeneity and stability thereof were confirmed.

### SUMMARY OF THE VARIETY

This new variety of Petunia plant ‘SUNBELBURA’ has a decumbent habit with very small pale purplish pink color petals with deep reddish purple vein flowers and thus is very different from a similar variety, ‘SUNBELKUPI’, and ‘Pearl Sky Blue’. The plant has a decumbent habit, abundant branching and a great profusion of blooms, with the whole plant remaining in bloom for a considerable period of time. The secondary branching is especially strong. The flower size is very small and the petal color of flower is pale purplish pink with deep reddish purple veins, which is clearly distinguished from the vivid reddish purple petals of ‘SUNBELKUPI’ and the strong bluish purple petals of ‘Pearl Sky Blue’. The new variety, ‘SUNBELBURA’, has a pale purplish pink petal coloration with deep reddish purple vein, a brilliant greenish yellow bottom color of the corolla throat and the outside color of the corolla tube is pale yellow green. The plant is highly resistance to cold, rain, heat, drought, pests and diseases. The resistance to rain and heat is very strong.

### BRIEF DESCRIPTION OF THE DRAWINGS

FIG. 1 is a photograph of flowers of the new variety of petunia plant.

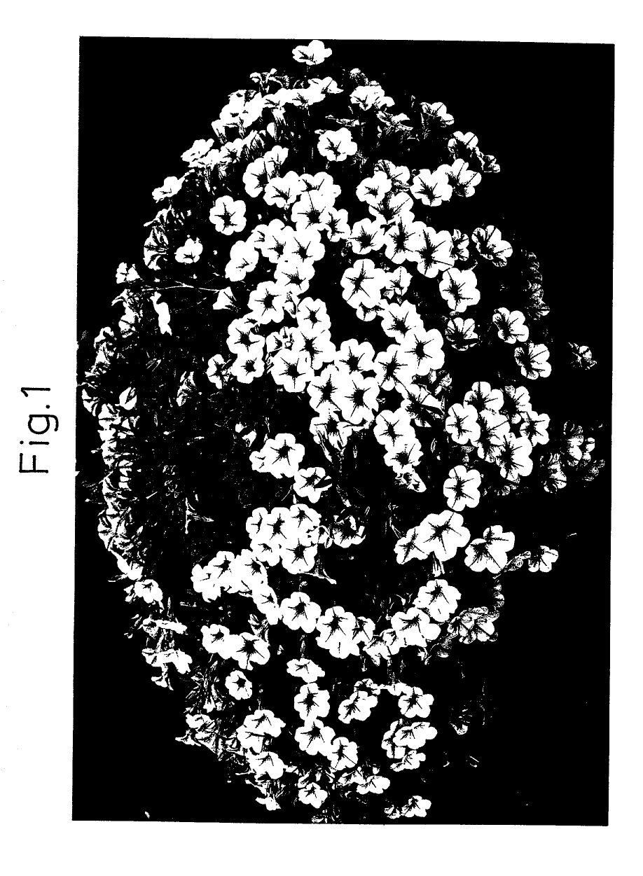

FIG. 2 is a photograph showing a partial view of the new variety of petunia plant planted in a flower pot.

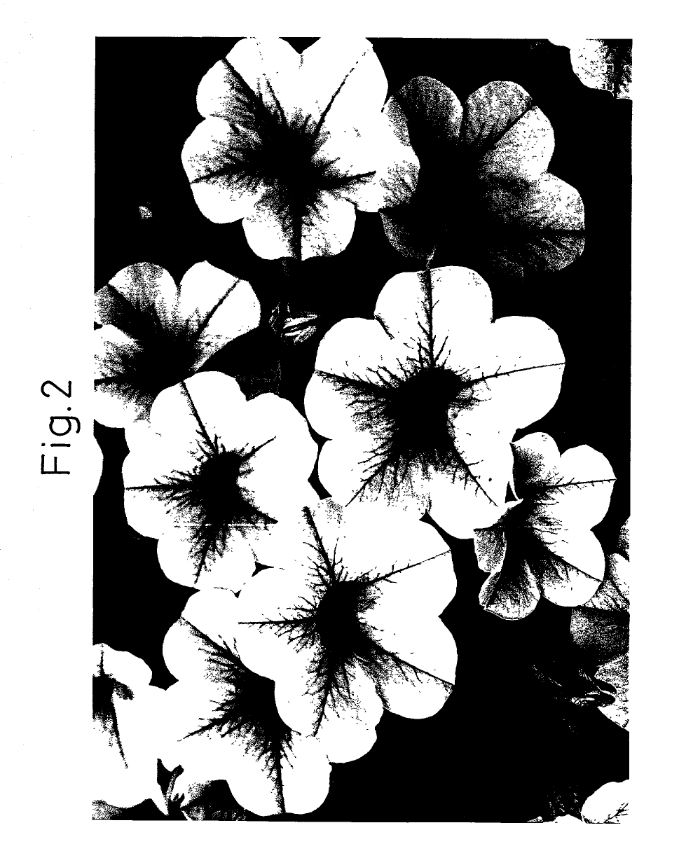

### DESCRIPTION OF THE NEW VARIETY

The botanical characteristics of the new and distinct variety of Petunia plant named ‘SUNBELBURA’ are as follows, from observation, during July at Yokaichi-shi, Shiga-ken, Japan, of young plants that had been reproduced by cutting during June the year before.

Plant:

Growth habit.—Decumbent.

Plant height.—very short; 7 cm.

Spreading area of plant.—The stem extends to a length of 20 cm from the base.

Growth.—Very vigorous with abundant branching, and a great profusion of blooms, with the whole bush remaining in bloom for a considerable period of time.

Blooming period.—May to October in the southern Kanto area, Japan. The plant shape does not change throughout this period.

Stem:

Thickness.—Thin; 1.7 mm.

Pubescence.—Sparse to normal.

Branching.—Medium with a superior branching propensity. The secondary branches are particularly numerous.

Length of internode.—Short; 1.4 cm.

Leaf:

Shape.—Lanceolate.

Length.—Very Short; 3.4 cm.

Width.—Very Narrow; 0.9 cm.

Color.—Moderate yellow green. (R.H.S. color chart No. 137B, JHS color chart No. 3712).

Thickness.—Thin; 0.5 mm.

Pubescence.—Sparse.

Leaf attachment angle to stem.—Slanted upward to horizontal.

Flower:

Facing direction.—Slanted upward to horizontal.

Type.—Single.

Shape.—Funnel-shape, with five-fissured limb.

Shape of petal tip.—Obverse.

Waving of petal.—Weak.

Lobation of petal.—Very shallow.

Diameter.—Very small; 2.9 mm.

Variegated pattern of corolla.—Vein.

Ground color of bi-colored petal.—Pale purplish pink (R.H.S. color chart No. 69B, JHS color chart No. 9202).

Color of variegated pattern.—Deep reddish purple (R.H.S. color chart No. 77A, JHS color chart No. 9209).

Bottom color of the corolla throat.—Brilliant greenish yellow (R.H.S. color chart No. 3A, JHS color chart No. 2905).

Outside color of corolla tube.—Pale yellow green (R.H.S. color chart No. 1D, JHS color chart No. 3102).

Reproductive organs.—1 normal pistil and 5 normal stamens.

Fertility.—Fertile, but self-incompatible.

Peduncle.—0.6 mm in thickness, and 1.5 cm in length.

Physiological and ecological characteristics.—High resistance to cold, rain, heat, drought and diseases such as Powdery Mildew. The resistance to rain and heat is very strong.

Blooming.—A bloom commonly lasts approximately 10 days on the plant. Pinching is not necessary to ensure continued blooming; however, it does tend to enhance bloom production somewhat.

This new variety of Petunia plant is most suitable for flower bedding and potting, particularly in hanging pots and planters, and is further excellent for use as ground cover.

The plant of this new variety, ‘SUNBELBURA’, is presently planted and maintained at Yokaichi-shi, Shiga-ken, Japan.

1. A new and distinct variety of Petunia plant, substantially as herein illustrated and described, characterized particularly as to novelty by (A) having a semi-erect habit, (B) abundant branching, the secondary branching being particularly strong, (C) a plentiful number of flowers in a spike and a great profusion blooms, with the entire plant remaining in bloom for a considerable period of time, (D) a very small flower size, the petals having a pale purplish pink color with deep reddish purple vein, (E) a high resistance to rain, cold, heat, drought, diseases and pests, and (F) a late flowering time.
